(19) United States
(12) Patent Application Publication
(10) Pub. No.: US 2009/0281078 A1
(43) Pub. Date: Nov. 12, 2009

(54) COMBINATION OF SABCOMELINE WITH A NEUROLEPTIC AGENT TO TREAT PSYCHOTIC DISORDERS

(76) Inventors: Carol ROUTLEDGE, London (GB); James Joseph Hagan, Essex (GB); Stuart Paul Cuffe, Research Triangle Park, NC (US)

Correspondence Address: NIXON & VANDERHYE, PC, 901 NORTH GLEBE ROAD, 11TH FLOOR, ARLINGTON, VA, 22203, US

(21) Appl. No.: 12/504,366

(22) Filed: Jul. 16, 2009

Related U.S. Application Data
(62) Division of application No. 11/792,849, filed on Oct. 31, 2007.

(30) Foreign Application Priority Data
Dec. 23, 2004 (GB) 0428180.4

(51) Int. Cl. A61K 31/554; A61K 31/439; A61K 31/519; A61K 31/496; A61P 25/00

(52) U.S. Cl. 514/211.13; 514/305; 514/259.41; 514/253.07; 514/254.04

(57) ABSTRACT

The invention relates to adjunctive and simultaneous combination therapies for the treatment of psychotic disorders in which sabcomeline or a pharmaceutically acceptable salt thereof and at least one other neuroleptic agent are administered adjunctively or simultaneously. The invention provides methods of treatment of psychotic disorders utilising such adjunctive or simultaneous therapeutic combination therapies, therapeutic combinations for use therein and pharmaceutical compositions comprising them.

This application is a divisional of application Ser. No. 11/792,849 (U.S. Patent Application Publication No. US-2008/0081804), filed Oct. 31, 2007 (pending), which is a U.S. national phase of International Application No. PCT/GB2005/005057 filed 23 Dec. 2005, which designated the U.S. and claims benefit of GB 0428180.4, filed 23 Dec. 2004, the entire contents of each of which is hereby incorporated by reference in this application.

This invention relates to combination therapy for treating psychotic and other mood disorders, to therapeutic combinations and compositions comprising them, and to methods of treatment of psychotic and other mood disorders.

U.S. Pat. No. 5,278,170 describes a class of compounds which enhance acetylcholine function via an action at muscarinic receptors within the central nervous system. A particularly preferred compound from within the scope of this disclosure has been given the common name sabcomeline, and has the following chemical structure (I)

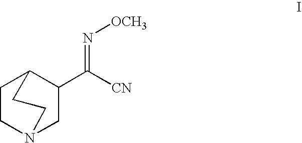

The chemical name for sabcomeline is R-(Z)-α-(methoxyimino)-α-(1-azabicyclo[2.2.2]oct-3-yl)acetonitrile. For therapeutic administration, it is preferably used in the form of a pharmaceutically acceptable salt, typically the hydrochloride salt, but alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, furmaric acid, maleic acid, salicylic acid, citric acid, lactic acid, oxalic acid and p-toluene sulphonic acid.

An example of the method of preparation of sabcomeline is as follows; to a stirred solution of potassium tert-butoxide (94.1 g; 0.84 mol) in tetrahydrofuran (250 ml) under nitrogen is added a solution of 3-(cyanomethyl)quinuclidine (60 g; 0.4 mol) in tetrahydrofuran (150 ml) during a period of 10 mins. The reaction is stirred for 10 minutes then cooled to 0° C. isoamyl nitrite (51.5 g 0.44 mol) is added at a rate such that the internal temperature does not exceed 25° C. The reaction is stirred for 20 minutes then diluted with dimethylsulphoxide (500 ml). Methyl tosylate (134 g; 0.72 mol) is added as a solution in dimethylsulphoxide (100 ml) at a rate such that the temperature does not exceed 35° C. After a further 20 minutes aqueous potassium carbonate (ca 5 wt % 500 ml) is added and the reaction extracted with ethyl acetate (5×200 ml). The ethyl acetate extract is washed with 5 wt % aqueous potassium carbonate (4×250 ml), then saturated potassium carbonate (50 ml). The combined aqueous layers are re-extracted with ethyl acetate (500 ml) which is washed as above. The combined organic extracts are dried over anhydrous potassium carbonate (200 g) and concentrated in vacuo to give a brown oil containing ca. 80 wt % 3-[(cyano)(methoxyimino)-methyl]quinuclidine as a 4:1 mixture of Z:E isomers, (47.4 g; 0.245 mol; 61%).

Alternative methods for producing pharmaceutically acceptable salts of sabcomeline including intermediates thereof are described in EP0626961 and are included herein by way of reference.

Sabcomeline was initially evaluated for its use in the treatment of dementia. Subsequently, a number of disclosures have disclosed the use of sabcomeline for treating psychotic disorders, for example WO 98/46226. WO 02/03684 further discloses the treatment of psychotic disorders by administration of a muscarinic agonist in combination with a typical or an atypical antipsychotic. Although sabcomeline is disclosed in WO 02/03684 as one of a number of muscarinic agonists suitable for combination with a large number of typical and atypical antipsychotics, exemplification is limited to just one muscarinic agonist (xanomeline) in combination with a small number of antipsychotics, and no specific information or data are recorded concerning combination therapy involving sabcomeline. There remains a need to identify further and improved medicaments for use in the treatment of psychotic disorders, and in particular compositions and methods of treatment which improve on the efficacy of existing neuroleptic therapies.

It has now been found that sabcomeline or a pharmaceutically acceptable salt thereof may advantageously be administered in combination with at least one neuroleptic agent to provide improved treatment of psychotic disorders. Particular advantages associated with the combinations, uses and methods of treatment of the invention include equivalent or improved efficacy at doses of administration which are lower than those commonly used for the individual components. Improved treatments of positive symptoms and/or negative symptoms and/or cognitive symptoms of the psychotic disorders may also be observed. The combinations, uses and methods of treatment of the invention may also provide advantages in treatment of patients who fail to respond adequately or who are resistant to treatment with certain neuroleptic agents.

The term neuroleptic refers to the effects on cognition and behavior of antipsychotic drugs that reduce confusion, delusions, hallucinations, and psychomotor agitation in patients with psychoses. Also known as major tranquilizers and antipsychotic drugs, neuroleptic agents comprise a group of the following 7 classes of drugs: Phenothiazines, further divided into the aliphatics, piperidines, and piperazines, Thioxanthenes (eg, droperidol), Butyrophenones (eg, haloperidol), Dibenzoxazepines (eg, loxapine), Dihydroindolone (eg, molindone), Diphenylbutylpiperidine (eg, pimozide), Benzisoxazole (eg, risperidone).

Antipsychotics can be classified by their structure but can also be distinguished by their pharmacology, their action at receptors, and by their clinical properties. Typical (also called conventional) antipsychotics act primarily at dopamine receptors. Atypical antipsychotics act on other receptors as well as dopamine, and are less likely than typical antipsychotics to cause movement disorders as a side effect. Examples of atypical antipsychotics include amisulpiride (brand name Solian®), aripiprazole (Abilify®), clozapine (Clozaril®), olanzapine (Zyprexa®), quetiapine (Seroquel®), risperidone (Risperdal®) and zotepine (Zoleptil®).

It is believed that the clinical utility of the combination of sabcomeline and antipsychotic may vary between different members of the atypical antipsychotic drug class, depending on their different affinities for various sub-types of neurochemical receptors. For example, in addition to their affinities for dopamine and serotonin receptors, members of the atypical antipsychotic class may vary in their affinity for muscarinic and histamine receptor sub-types. The activity of atypical neuroleptics at muscarinic receptor subtypes are such that properties of negligible affinity, weak agonist activity and weak antagonist activity have been reported amongst the various members of the atypical antipsychotic drug class.

As an example, the M1/M4 receptor agonist properties of sabcomeline may enhance functional cholinergic activity and, when administered in combination, provide benefit by:

i) enhancing functional cholinergic activity in combination with an atypical antipsychotic that itself has little or no affinity for muscarinic receptors (e.g. risperidone)

ii) providing additive functional cholinergic activity in combination with an atypical antipsychotic drug that has weak muscarinic receptor agonist effects (e.g. clozapine or N-desmethylclozapine)

iii) competing for muscarinic receptors and thereby reducing the anticholinergic functional effects of an atypical antipsychotic drug that possesses muscarinic receptor antagonist properties (e.g. olanzepine ).

As well as muscarinic and histaminergic receptors there are other receptors that may have benefit or adverse effects on cognition. For instance drugs with 5-HT6 receptor antagonist and adrenergic α2 receptor antagonist properties may also be of benefit. Some atypicals also have these benefits.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of sabcomeline or a pharmaceutically acceptable salt thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is preferred that sabcomeline or a pharmaceutically acceptable salt thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of sabcomeline or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a first aspect therefore, the invention provides a method of treatment of a psychotic disorder by adjunctive therapeutic administration of sabcomeline or a pharmaceutically acceptable salt thereof to a patient receiving therapeutic administration of at least one neuroleptic agent. In a further aspect, the invention provides the use of sabcomeline or a pharmaceutically acceptable salt thereof in the manufacture of a medicament for adjunctive therapeutic administration for the treatment of a psychotic disorder in a patient receiving therapeutic administration of at least one neuroleptic agent. The invention also provides the use of sabcomeline or a pharmaceutically acceptable salt thereof in adjunctive therapeutic administration for the treatment of a psychotic disorder in a patient receiving therapeutic administration of at least one neuroleptic agent. The invention further provides sabcomeline or a pharmaceutically acceptable salt thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of sabcomeline or a pharmaceutically acceptable salt thereof. In a further aspect, the invention provides the use of at least one neuroleptic agent in the manufacture of a medicament for adjunctive therapeutic administration for the treatment of a psychotic disorder in a patient receiving therapeutic administration of sabcomeline or a pharmaceutically acceptable salt thereof. The invention also provides the use of at least one neuroleptic agent for adjunctive therapeutic administration for the treatment of a psychotic disorder in a patient receiving therapeutic administration of sabcomeline or a pharmaceutically acceptable salt thereof.

In a further aspect, the invention provides a method of treatment of a psychotic disorder by simultaneous therapeutic administration of sabcomeline or a pharmaceutically acceptable salt thereof in combination with at least one neuroleptic agent. The invention further provides the use of a combination of sabcomeline or a pharmaceutically acceptable salt thereof and at least one neuroleptic agent in the manufacture of a medicament for simultaneous therapeutic administration in the treatment of a psychotic disorder. The invention further provides the use of a combination of sabcomeline or a pharmaceutically acceptable salt thereof and at least one neuroleptic agent for simultaneous therapeutic administration in the treatment of a psychotic disorder. The invention further provides the use of sabcomeline or a pharmaceutically acceptable salt thereof in the manufacture of a medicament for simultaneous therapeutic administration with at least one neuroleptic agent in the treatment of a psychotic disorder. The invention further provides the use of sabcomeline or a pharmaceutically acceptable salt thereof for simultaneous therapeutic administration with at least one neuroleptic agent in the treatment of a psychotic disorder. The invention further provides sabcomeline or a pharmaceutically acceptable salt thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with sabcomeline or a pharmaceutically acceptable salt thereof in the treatment of a psychotic disorder. The invention further provides the use of at least one neuroleptic agent for simultaneous therapeutic administration with sabcomeline or a pharmaceutically acceptable salt thereof in the treatment of a psychotic disorder.

In further aspects, the invention provides a method of treatment of a psychotic disorder by simultaneous therapeutic administration of a pharmaceutical composition comprising sabcomeline or a pharmaceutically acceptable salt thereof and at least one mood stabilising or antimanic agent, a pharmaceutical composition comprising sabcomeline or a pharmaceutically acceptable salt thereof and at least one mood stabilising or antimanic agent, the use of a pharmaceutical composition comprising sabcomeline or a pharmaceutically acceptable salt thereof and at least one mood stabilising or antimanic agent for the treatment of a psychotic disorder, the use of a pharmaceutical composition comprising sabcomeline or a pharmaceutically acceptable salt thereof and at least one mood stabilising or antimanic agent in the manufacture of a medicament for the treatment of a psychotic disorder, and a pharmaceutical composition comprising sabcomeline or a pharmaceutically acceptable salt thereof and at least one mood stabilising or antimanic agent for use in the treatment of a psychotic disorder.

In a further aspect, the invention provides a kit-of-parts for use in the treatment of a psychotic disorder comprising a first dosage form comprising sabcomeline or a pharmaceutically acceptable salt thereof and one or more further dosage forms each comprising a neuroleptic agent for simultaneous therapeutic administration.

Within the context of the present invention, the term psychotic disorder includes schizophrenia, schizophreniform diseases, schizoaffective disorders, delusional disorders, effective disorders, autism, tic disorders, depression with psychotic features, chronic schizophrenic psychoses, schizoaffective psychoses, and temporary acute psychotic disorders. The above mentioned conditions represent multiple disease states. For example, schizophrenia is referred to in various forms as catatonic, disorganised, paranoid, undifferential, residual, among others. All the various forms of the disorders mentioned herein are contemplated as part of the present invention.

The following list further illustrates a number of these disease states, many of which are classified in the Diagnostic and Statistical Manual of Mental Disorders, 4^{th} Edition, published by the American Psychiatric Association (DSM IV): Paranoid Type Schizophrenia, Disorganised Type Schizophrenia, Catatonic Type Schizophrenia, Undifferentiated Type Schizophrenia, Residual Type Schizophrenia, Schizophreniform Disorder, Schizoaffective Shared Psychotic Disorder, Psychotic Disorder Due to a General Medical Condition, Substance-induced Psychotic Disorder, Psychotic Disorder with Psychotic Features, Schizoid Personality Disorder and Schizotypal Personality Disorder. The list also includes forms of schizophrenia which are resistant to methods and means of treatment of the prior art.

The treatment of psychotic disorders with sabcomeline or a pharmaceutically acceptable salt thereof and a neuroleptic agent as defined in the present invention may occur in addition to further drug therapies. In particular, tranquilizers may be used for the treatment of agitation, anxiety or sleep disturbances. Preferably lorazepam is used, which belongs to the class of benzodiazepines. Antidepressants and anxiolytics may also be used, for example SSRI antidepressants such as paroxetine or fluoxetine.

Examples of neuroleptic agents that are useful in the present invention include, but are not limited to: butyrophenones, such as haloperidol, pimozide, and droperidol; phenothiazines, such as chlorpromazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazines such as lamotrigine; dibenzoxazepines, such as loxapine; dihydroindolones, such as molindone; aripiprazole; and derivatives thereof that have antipsychotic activity. Further examples of neuroleptic agents that may be used in the present invention include carbamazepine, valproate, gabapentin, topiramate, oxcarbazepine and lithium. Particular examples of neuroleptic agents and their typical route of administration and dosage ranges that are preferred for use in the present invention are shown in Table 1.

TABLE 1
Neuroleptic agents

| | | | | Dosage | |
|---|---|---|---|---|---|
| Common | | Route of | | Range and | |
| Name | Trade Name | Administration | Form | (Median)^{a} | |
| Clozapine | CLOZARIL | oral | tablets | 12.5-900 | mg/day |
| | | | | (300-900 | mg/day) |
| Olanzapine | ZYPREXA | oral | tablets | 5-25 | mg/day |
| | | | | (10-25 | mg/day) |
| Ziprasidone | GEODON | oral | capsules | 20-80 | mg/twice a day |
| | | | | (80-160 | mg/day) |
| Risperidone | RISPERDAL | oral | solution tablets | 2-16 | mg/day tablets |
| | | | | (4-12 | mg/day) |
| Risperidone | RISPERDAL | Intra-venous | Long-acting | | |
| | | | injectable form | | |
| Quetiapine | SEROQUEL | oral | tablets | 50-900 | mg/day |
| fumarate | | | | (300-900 | mg/day) |
| Sertindole | SERDILECT | | | (4-24 | mg/day) |
| Amisulpiride | | | | | |
| Sulpiride | | | | | |
| Haloperidol | HALDOL | oral | tablets | 1-100 | mg/day |
| | | | | (1-15 | mg/day) |
| Haloperidol | HALDOL | parenteral | injection | | |
| Decanoate | Decanoate | | | | |
| Haloperidol | HALDOL | oral | solution | | |
| lactate | INTENSOL | parenteral | injection | | |
| Chlorpromazine | THORAZINE | rectal | suppositories | 30-800 | mg/day |
| | | oral | capsules | (200-500 | mg/day) |
| | | | solution | | |
| | | | tablets | | |
| | | parenteral | injection | | |
| Fluphenazine | PROLIXIN | | | 0.5-40 | mg/day |
| | | | | (1-5 | mg/day) |
| Fluphenazine | PROLIXIN | parenteral | injection | (about one-half | |
| decanoate | Decanoate | | | the dosage | |
| | | | | shown for oral) | |
| Fluphenazine | PROLIXIN | parenteral | injection | (same as above | |
| enanthate | | | | | |
| Fluphenazine | PROLIXIN | oral | elixer | | |
| hydrochloride | | | solution | | |
| | | parenteral | injection | | |
| Thiothixene | NAVANE | oral | capsules | 6-60 | mg/day |
| | | | | (8-30 | mg/day) |
| Thiothixene | NAVANE | oral | solution | | |
| hydrochloride | | parenteral | injection | | |
| Trifluoperazine | STELAZINE | | | (2-40 | mg/day) |
| Perphenazine | TRILAFON | oral | solution | 12-64 | mg/day |
| | | | tablets | (16-64 | mg/day) |
| | | parenteral | injection | | |
| Perphenazine | ETRAFON | oral | tablets | | |
| and | TRIAVIL | | | | |
| Amitriptyline | | | | | |
| hydrochloride | | | | | |
| Thioridazine | MELLARIL | Oral | Suspension | 150-800 | mg/day |
| | | | Solution | (100-300 | mg/day) |
| | | | Tablets | | |
| Mesoridazine | | | | (30-400 | mg/day) |
| Molindone | MOBAN | | | 50-225 | mg/day |
| | | | | (15-150 | mg/day) |
| Molindone | MOBAN | oral | solution | | |
| hydrochloride | | | | | |
| Loxapine | LOXITANE | | | 20-250 | mg/day |
| | | | | (60-100 | mg/dav) |
| Loxapine | LOXITANE | oral | solution | | |
| hydrochloride | | parenteral | injection | | |
| Loxapine | LOXITANE | oral | capsules | | |
| succinate | | | | | |
| Pimozide | | | | (1-10 | mg/day) |
| Flupenthixol | | | | | |
| Promazine | SPARINE | | | | |
| Triflupromazine | VESPRIN | | | | |
| Chlorprothixene | TARACTAN | | | | |
| Droperidol | INAPSINE | | | | |
| Acetophenazine | TINDAL | | | | |
| Prochlorperazine | COMPAZINE | | | | |
| Methotrimeprazine | NOZINAN | | | | |
| Pipotiazine | PIPOTRIL | | | | |
| Aripiprazole | ABILIFY | | | | |
| Hoperidone | | | | | |

Examples of tradenames and suppliers of selected neuroleptic agents are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from GlaxoSmithKline; fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from GlaxoSmithKline; perphenazine (available under the tradename TRILAFON®; from Schering); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Particularly preferred neuroleptic agents for use in the invention are olanzapine, risperidone, quetiapine, aripiprazole, haloperidol, clozapine, ziprasidone and osanetant.

A particularly preferred aspect of the invention provides a method of treatment of a psychotic disorder by adjunctive therapeutic administration of sabcomeline or a pharmaceutically acceptable salt thereof to a patient receiving administration of olanzapine. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of olanzapine. A further preferred aspect of the invention provides the use of sabcomeline or a pharmaceutically acceptable salt thereof for adjunctive therapeutic administration for the treatment of a psychotic disorder in a patient receiving administration of olanzapine. A further preferred aspect of the invention provides sabcomeline or a pharmaceutically acceptable salt thereof for use for adjunctive therapeutic administration for the treatment of a psychotic disorder in a patient receiving administration of olanzapine.

A particularly preferred aspect of the invention provides a method of treatment of a psychotic disorder by adjunctive therapeutic administration of sabcomeline or a pharmaceutically acceptable salt thereof to a patient receiving administration of risperidone. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of risperidone. A further preferred aspect of the invention provides the use of sabcomeline or a pharmaceutically acceptable salt thereof for adjunctive therapeutic administration for the treatment of a psychotic disorder in a patient receiving administration of risperidone. A further preferred aspect of the invention provides sabcomeline or a pharmaceutically acceptable salt thereof for use for adjunctive therapeutic administration for the treatment of a psychotic disorder in a patient receiving administration of risperidone.

A particularly preferred aspect of the invention provides a method of treatment of a psychotic disorder by adjunctive therapeutic administration of sabcomeline or a pharmaceutically acceptable salt thereof to a patient receiving administration of quetiapine. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of quetiapine. A further preferred aspect of the invention provides the use of sabcomeline or a pharmaceutically acceptable salt thereof for adjunctive therapeutic administration for the treatment of a psychotic disorder in a patient receiving administration of quetiapine. A further preferred aspect of the invention provides sabcomeline or a pharmaceutically acceptable salt thereof for use for adjunctive therapeutic administration for the treatment of a psychotic disorder in a patient receiving administration of quetiapine.

A particularly preferred aspect of the invention provides a method of treatment of a psychotic disorder by adjunctive therapeutic administration of sabcomeline or a pharmaceutically acceptable salt thereof to a patient receiving administration of aripiprazole. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of aripiprazole. A further preferred aspect of the invention provides the use of sabcomeline or a pharmaceutically acceptable salt thereof for adjunctive therapeutic administration for the treatment of a psychotic disorder in a patient receiving administration of aripiprazole. A further preferred aspect of the invention provides sabcomeline or a pharmaceutically acceptable salt thereof for use for adjunctive therapeutic administration for the treatment of a psychotic disorder in a patient receiving administration of aripiprazole.

A particularly preferred aspect of the invention provides a method of treatment of a psychotic disorder by adjunctive therapeutic administration of sabcomeline or a pharmaceutically acceptable salt thereof to a patient receiving administration of haloperidol. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of haloperidol. A further preferred aspect of the invention provides the use of sabcomeline or a pharmaceutically acceptable salt thereof for adjunctive therapeutic administration for the treatment of a psychotic disorder in a patient receiving administration of haloperidol. A further preferred aspect of the invention provides sabcomeline or a pharmaceutically acceptable salt thereof for use for adjunctive therapeutic administration for the treatment of a psychotic disorder in a patient receiving administration of haloperidol.

A particularly preferred aspect of the invention provides a method of treatment of a psychotic disorder by adjunctive therapeutic administration of sabcomeline or a pharmaceutically acceptable salt thereof to a patient receiving administration of clozapine. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of clozapine. A further preferred aspect of the invention provides the use of sabcomeline or a pharmaceutically acceptable salt thereof for adjunctive therapeutic administration for the treatment of a psychotic disorder in a patient receiving administration of clozapine. A further preferred aspect of the invention provides sabcomeline or a pharmaceutically acceptable salt thereof for use for adjunctive therapeutic administration for the treatment of a psychotic disorder in a patient receiving administration of clozapine.

A particularly preferred aspect of the invention provides a method of treatment of a psychotic disorder by adjunctive therapeutic administration of sabcomeline or a pharmaceutically acceptable salt thereof to a patient receiving administration of ziprasidone. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of ziprasidone. A further preferred aspect of the invention provides the use of sabcomeline or a pharmaceutically acceptable salt thereof for adjunctive therapeutic administration for the treatment of a psychotic disorder in a patient receiving administration of ziprasidone. A further preferred aspect of the invention provides sabcomeline or a pharmaceutically acceptable salt thereof for use for adjunctive therapeutic administration for the treatment of a psychotic disorder in a patient receiving administration of ziprasidone.

A particularly preferred aspect of the invention provides a method of treatment of a psychotic disorder by adjunctive therapeutic administration of sabcomeline or a pharmaceutically acceptable salt thereof to a patient receiving administration of osanetant. A further preferred aspect of the invention provides the use of sabcomeline or a pharmaceutically acceptable salt thereof in the manufacture of a medicament for adjunctive therapeutic administration for the treatment of a psychotic disorder in a patient receiving administration of osanetant. A further preferred aspect of the invention provides the use of sabcomeline or a pharmaceutically acceptable salt thereof for adjunctive therapeutic administration for the treatment of a psychotic disorder in a patient receiving administration of osanetant. A further preferred aspect of the invention provides sabcomeline or a pharmaceutically acceptable salt thereof for use for adjunctive therapeutic administration for the treatment of a psychotic disorder in a patient receiving administration of osanetant.

For therapeutic administration according to the present invention, the sabcomeline component may be employed in the form of its free base, but is preferably used in the form of a pharmaceutically acceptable salt, typically the hydrochloride salt. Alternative salts of sabcomeline with pharmaceutically acceptable acids may also be utilised in therapeutic administration, for example salts derived from sabcomeline or a pharmaceutically acceptable salt thereof free base and acids including, but not limited to, hydrobromic acid, phosphoric acid, acetic acid, furmaric acid, maleic acid, salicylic acid, citric acid, oxalic acid, lactic acid, malic acid, methanesulphonic acid and p-toluene sulphonic acid. All solvates and all alternative physical forms of sabcomeline or its pharmaceutically acceptable derivatives as described herein, including but not limited to alternative crystalline forms, amorphous forms and polymorphs are also within the scope of this invention, and all references to sabcomeline herein include all pharmaceutically acceptable salts, and all solvates and alternative physical forms thereof.

The neuroleptic agent component or components may also be administered in their basic or acidic forms as appropriate or, where appropriate, in the form of a pharmaceutically acceptable salt or other derivative. All solvates and all alternative physical forms of the neuroleptic agent or agents or their pharmaceutically acceptable salts or derivatives as described herein, including but not limited to alternative crystalline forms, amorphous forms and polymorphs, are also within the scope of this invention. In the case of the neuroleptic agent or agents, the preferred forms and derivatives are those which are approved for therapeutic administration as monotherapies, including those mentioned in Table I, but all references to neuroleptic agents herein include all pharmaceutically acceptable salts or other derivatives thereof, and all solvates and alternative physical forms thereof.

For adjunctive or simultaneous therapeutic administration according to the invention, sabcomeline or its pharmaceutically acceptable salts or solvates and the neuroleptic agent or agents or their pharmaceutically acceptable salts, derivatives or solvates may each be administered in pure form, but each of the components will preferably be formulated into any suitable pharmaceutically acceptable and effective composition which provides effective levels of the respective component in the body. The choice of the most appropriate pharmaceutical compositions for each component is within the skill of the art, and may be the same form or different forms for each of the components. Suitable formulations include, but are not limited to tablets, capsules, powders, granules, lozenges, suppositories, reconstitutable powders, or liquid preparations such as oral or sterile parenteral solutions or suspensions.

For simultaneous administration as a combined composition of sabcomeline and the neuroleptic agent or agents according to the invention, sabcomeline or its pharmaceutically acceptable salts or solvates and the neuroleptic agent or agents and their pharmaceutically acceptable salts, derivatives or solvates may be administered together in pure form, but the combined components will preferably be formulated into any suitable pharmaceutically acceptable and effective composition which provides effective levels of each of the components in the body. The choice of the most appropriate pharmaceutical compositions for the combined components is within the skill of the art. Suitable formulations include, but are not limited to tablets, sub-lingual tablets, buccal compositions, capsules, powders, granules, lozenges, suppositories, reconstitutable powders, or liquid preparations such as oral or sterile parenteral solutions or suspensions.

In order to obtain consistency of adjunctive administration or simultaneous administration, it is preferred that the compositions of each of the components, or of the combination of the components is in the form of a unit dose.

Unit dose presentation forms of the components, or of the combination of the components, for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of biending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations for the components, or for the combination of the components, may be in the form of, for example, emulsions, syrups, suspensions or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration (for example intravenous, intravascular or subcutaneous administration) of the components, or of the combination of the components, fluid unit dosage forms are prepared utilizing the component or the combination of the components and a sterile vehicle, and, depending on the concentration used, can be either suspended or dissolved in the vehicle. In preparing solutions the components or the combination of the components can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the component is suspended in the vehicle instead of being dissolved, and sterilization cannot be accomplished by filtration. The components, or the combination of the components can be sterilized by exposure to ethylene oxide before suspending in the sterile vehicle. Advantageously, a surfactant or wetting agent is included in the composition to facilitate uniform distribution of the component or the combination of the components.

The components or the combination of the components may also be formulated as depot preparations. Such long acting formulations may be administered by implantation (for example subcutaneously or intramuscularly) or by intramuscular injection. Thus, for example, the components or the combination of the components of the invention may be formulated with suitable polymeric or hydrophobic materials (for example as an emulsion in an acceptable oil) or ion exchange resins, or as sparingly soluble derivatives, for example, as a sparingly soluble salt.

The compositions of each of the components or of the combination or of the components may contain from 0.1% to 99% by weight, preferably from 10-60% by weight, of the active material, depending on the method of administration.

For adjunctive or simultaneous administration, the unit dose of the sabcomeline component is in the range of 10-300 microgrammes, each unit dose being administered up to four times daily. Preferably the unit dose of the sabcomeline component is in the range 25-100 microgrammes, each unit dose being administered up to four times daily. The daily and unit doses of the neuroleptic agent will depend upon which neuroleptic agent is employed, but will typically be the recommended or approved dosage for the specific neuroleptic agent when administered as monotherapy. In a preferred aspect of the invention, adjunctive administration of sabcomeline may permit lower doses of the neuroleptic agent than those normally recommended when the neuroleptic agent is prescribed as monotherapy. Typical daily doses of the neuroleptic agents suitable for use in adjunctive or simultaneous administration according to the invention are shown in Table 1.

The adjunctive or simultaneous administration of at least one neuroleptic agent and sabcomeline as described herein may also be useful in the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. Other mood disorders encompassed within the term major depressive disorders include dysthymic disorder with early or late onset and with or without atypical features, neurotic depression, post traumatic stress disorders, post operative stress and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

The adjunctive or simultaneous administration of at least one neuroleptic agent and sabcomeline as described herein may also be useful in the treatment of sleep disorders including dysomnia, insomnia, sleep apnea, narcolepsy, and circadian rhythmic disorders.

The adjunctive or simultaneous administration of at least one neuroleptic agent and sabcomeline as described herein may also be useful in the treatment of tolerance to and dependence on a number of substances. For example, in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative hypnotic, amphetamine or amphetamine-related drugs (e.g. dextroamphetamine, methylamphetamine) addiction or a combination thereof.

The invention may be illustrated by suitable patient studies. The following example of a suitable patient study is for illustrative purposes and is not intended to limit the scope of the invention in any way. The study is a double-blind, placebo-controlled, randomised study of the efficacy of sabcomeline as therapy administered adjunctively to each of three neuroleptic agents for the treatment of cognitive effects in schizophrenia and schizoaffective disorder.

Approximately 50 patients aged 18-55 years with schizophrenia and schizoaffective disorder (as diagnosed using criteria defined in the DSM-IV) with cognitive deficits are selected. Eligible patients are those stabilised on neuroleptic agents for three months prior to the screening visit. The neuroleptic agents on which the patients in the illustrative trial are stabilised are haloperidol, risperidone or olanzapine. The selected patients are randomised to receive the previously administered neuroleptic agent plus placebo and the previously administered neuroleptic agent plus sabcomeline (50 microgrammes bid as the hydrochloride salt) for 12 weeks. After 12 weeks of treatment, the medication is suspended and patients return for consultation 2 weeks later. The neurocognitive effects of sabcomeline as adjunctive therapy to haloperidol, risperidone and olanzapine are evaluated using the Cogtest (Cognitive Function Test) battery as measured by Neurocognitive Global Score (NGS) and compared to those of the haloperidol, risperidone and olanzapine plus placebo. Change in PANSS (total Positive and Negative Syndrome Scale), CDSS (Calgary Depression Scale for Schizophrenia) and CGI (Clinical Global Impression Improvement) from baseline to end of study are also assessed. Alternatively, cogtest batteries such as Bacs and matrics may also be used. Additionally, it is anticipated there would be a second primary end-point addressing social functioning. An optimal duration to carry out studies and tests is 6 months.

1. A method of treating the cognitive and negative symptoms associated with schizophrenia comprising adjunctive therapeutic administration of sabcomeline or a pharmaceutically acceptable salt thereof to patients stabilized on amisulpride, risperidone, aripiprazole, ziprazidone or quetiapine.

2. A method of treatment of a psychotic disorder by administration of a pharmaceutical composition comprising sabcomeline or a pharmaceutically acceptable salt thereof and at least one neuroleptic agent, wherein the neuroleptic agent is an atypical antipsychotic selected from amisulpride, aripiprazole, quetiapine, risperidone and zotepine, to a patient.

3. A method of treatment according to claim 2 comprising adjunctive therapeutic administration of sabcomeline or a pharmaceutically acceptable salt thereof to a patient receiving therapeutic
administration of at least one neuroleptic agent.

4. A method of treatment according to claim 2 comprising adjunctive therapeutic administration of at least one neuroleptic agent to a patient receiving therapeutic administration of sabcomeline or a pharmaceutically acceptable salt thereof.

5. A method of treatment according to claim 2 comprising simultaneous therapeutic administration of sabcomeline or a pharmaceutically acceptable salt thereof in combination with at least one neuroleptic agent.

6. A method of claim 1 wherein the sabcomeline or a pharmaceutically acceptable salt thereof is administered in unit dosages of 10-300 micrograms up to 4 times per day.

7. A method of claim 2 wherein the sabcomeline or a pharmaceutically acceptable salt thereof is administered in unit dosages of 10-300 micrograms up to 4 times per day.
